(19) United States
(12) Patent Application Publication
(10) Pub. No.: US 2026/0306787 A1
(43) Pub. Date: Oct. 8, 2026

(54) SYSTEMS AND METHODS FOR SOLVING CAMERA POSE RELATIVE TO WORKING CHANNEL TIP

(71) Applicant: Covidien LP, Mansfield, MA (US)

(72) Inventor: Guy ALEXANDRONI, Yehud-Monosson (IL)

(21) Appl. No.: 19/681,633

(22) Filed: May 19, 2026

Related U.S. Application Data
(63) Continuation of application No. PCT/IB2024/061508, filed on Nov. 18, 2024.
(60) Provisional application No. 63/603,462, filed on Nov. 28, 2023.

(51) Int. Cl. A61M 25/00; A61B 1/04; A61B 34/20

(52) U.S. Cl. CPC A61M 25/00; A61B 1/042; A61B 34/20; A61B 2034/2065; A61M 2025/0004

(57) ABSTRACT

A system for performing a surgical procedure includes a first catheter defining a channel and an aperture having an inner dimension that is less than an inner dimension of the catheter, a transition portion disposed on an inner surface of the channel and adjacent to the aperture, and a pattern disposed on the transition portion storing positional information correlating a location on the pattern to a location on the first catheter, a second catheter receivable within the channel and having a camera, and a workstation operably coupled to the first and second catheters and including a processor and a memory storing instructions thereon, which when executed by the processor cause the processor to receive images captured by the camera and analyze the pattern visible within the received images to determine a position of the second catheter relative to the fist catheter.

### CROSS-REFERENCE TO RELATED APPLICATIONS

This application is a continuation of International Patent Application No. PCT/IB2024/061508, filed Nov. 18, 2024, which claims priority to U.S. Provisional Patent Application Serial No. 63/603,462, filed Nov. 28, 2023, the entire contents of each of which applications are incorporated herein by reference. To the extent appropriate, a claim of priority is made to each of the above disclosed applications.

### BACKGROUND

### Technical Field

The present disclosure relates to the field of navigating medical devices within a patient, and in particular, identifying a position of a medical device within a luminal network of a patient and navigating medical devices to a target.

### Description of the Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), cone-beam computed tomography (CBCT) or fluoroscopy (including 3D fluoroscopy) are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

However, access devices having an integrated camera may have difficulty navigating to the area of interest if the area of interest is located adjacent to small or narrow airways. As can be appreciated, the difficulty of navigating larges access devices, such as those having a camera, within narrow airways can lead to increased surgical times to navigate to the proper position relative to the area of interest which can lead to navigational inaccuracies or the use of fluoroscopy that leads to additional set-up time and radiation exposure. Further, target tissue often is not visible to white light cameras due to being located behind tissue walls or fluids, such as those resulting from biopsy or treatment activities that create bleeding and other occlusions.

### SUMMARY

A system for performing a surgical procedure includes a first catheter, the first catheter including an inner surface defining a channel extending through a proximal end portion of the first catheter and a distal end portion of the first catheter, an aperture disposed adjacent to the distal end portion of the first catheter, the aperture including an inner dimension that is less than an inner dimension of the channel, wherein the aperture is in open communication with the channel, a transition portion disposed on the inner surface of the channel and adjacent to the aperture, wherein the transition portion includes an inner dimension that increases from the inner dimension of the aperture to the inner dimension of the channel in a distal to proximal direction, and a pattern disposed on the transition portion, the pattern storing positional information correlating a location on the pattern to a location on the first catheter, a second catheter receivable within the channel, the second catheter including a camera having a field of view encompassing the pattern of the first catheter, and a workstation operably coupled to the first catheter and the second catheter, the workstation including a processor a memory storing thereon instructions, which when executed by the processor cause the processor to receive images captured by the camera of the second catheter, the pattern being visible within the received images, and analyze the pattern visible within the received images to determine a position of the second catheter relative to the first catheter.

In aspects, the second catheter may include an outer dimension that is greater than the inner dimension of the aperture to inhibit distal advancement of the second catheter past the aperture.

In certain aspects, the transition portion may define a tapered surface extending towards an inner portion of the channel.

In other aspects, the pattern may be selected from the group consisting of a one-dimensional barcode, DataMatrix, Maxicode, PDF417, a QR Code, a three-dimensional barcode, and a PM code.

In certain aspects, the positional information may be a rotational position and a longitudinal position.

In aspects, the first catheter may include a positional sensor, wherein the positional sensor is disposed a predetermined distance from the distal end portion of the first catheter.

In other aspects, the location on the first catheter may be a location of the positional sensor.

In certain aspects, the positional sensor may be an electromagnetic sensor.

In other aspects, the positional sensor may be an inertial measurement unit.

In aspects, the pattern may be etched into a surface of the transition portion.

In accordance with another aspect of the present disclosure, a catheter includes a proximal end portion, a distal end portion, an inner surface, the inner surface defining a channel extending through the proximal end portion and the distal end portion, an aperture disposed adjacent to the distal end portion, the aperture including an inner dimension that is less than an inner dimension of the channel, wherein the aperture is in open communication with the channel, a transition portion disposed on the inner surface of the channel and adjacent to the aperture, wherein the transition portion includes an inner dimension that increases from the inner dimension of the aperture to the inner dimension of the channel in a distal to proximal direction, and a pattern disposed on the transition portion, the pattern storing positional information correlating a location on the pattern to a location on the catheter.

In aspects, the pattern may be selected from the group consisting of a one-dimensional barcode, DataMatrix, Maxicode, PDF417, a QR Code, a three-dimensional barcode, and a PM code.

In other aspects, the positional information may be a rotational position and a longitudinal position.

In certain aspects, the catheter may include a positional sensor, wherein the positional sensor is disposed a predetermined distance from the distal end.

In aspects, the location on the catheter may be a location of the positional sensor.

In accordance with another aspect of the present disclosure, a method of navigating a medical device within a luminal network of a patient includes advancing a first catheter within a patient’ luminal network, advancing a second catheter within a channel defined through the first catheter, capturing images from a camera disposed on the second catheter, the captured images including a view of a pattern disposed on a portion of an inner surface of the channel of the first catheter, wherein the pattern stores positional information correlating a location on the pattern to a location on the first catheter, and analyzing the pattern visible within the captured images to determine a position of the second catheter relative to the location on the first catheter.

In certain aspects, analyzing the pattern visible within the captured images may include analyzing the pattern visible within the captured images to determine a rotational position, such as for example, a pose, of a distal portion of the second catheter and a longitudinal position of the distal portion of the second catheter relative to the location on the first catheter.

In other aspects, analyzing the pattern visible within the captured images may include analyzing the pattern visible within the captured images to determine the position of the second catheter relative to a positional sensor disposed on the first catheter, wherein the positional sensor is disposed a predetermined distance from a distal end portion of the first catheter.

In certain aspects, analyzing the pattern visible within the captured images may include analyzing a pattern selected from the group consisting of a one-dimensional barcode, DataMatrix, Maxicode, PDF417, a QR Code, a three-dimensional barcode, and a PM code.

In aspects, analyzing the pattern visible within the captured images may include analyzing the pattern visible within the captured images to determine a position of the second catheter relative to a positional sensor disposed at the location on the first catheter.

### BRIEF DESCRIPTION OF THE DRAWINGS

Various aspects and embodiments of the disclosure are described hereinbelow with references to the drawings, wherein:

FIG. 1 is a schematic view of a surgical system provided in accordance with the disclosure;

FIG. 2 is a cross-sectional view of a distal portion of a first catheter of the surgical system of FIG. 1;

FIG. 3 is a perspective view of a second catheter of the surgical system of FIG. 1;

FIG. 3A is a perspective view of another embodiment of the second catheter of FIG. 1;

FIG. 4 is a partial cross-sectional view of the first catheter of FIG. 2 showing the second catheter of FIG. 3 advanced within the first catheter;

FIG. 5 is a view through the distal end of the first catheter as observed and imaged by the second catheter;

FIG. 6 is a schematic view of a workstation of the surgical system of FIG. 1;

FIG. 7A is a flow diagram of a method of navigating a medical device to an area of interest within a patient’s luminal network;

FIG. 7B is a continuation of the flow diagram of FIG. 7A;

FIG. 8 is a perspective view of a robotic surgical system of the surgical system of FIG. 1; and

FIG. 9 is an exploded view of a drive mechanism of an extended working channel of the surgical system of FIG. 1.

### DETAILED DESCRIPTION

The disclosure is directed to a surgical system configured to enable navigation of a medical device through a luminal network of a patient, such as for example the lungs. The surgical system generates a 3-dimensional (3D) representation of the airways of the patient using pre-procedure images, such as for example, CT, CBCT, or MRI images and identifies an area of interest or target tissue within the 3D representation. The surgical system includes a bronchoscope, through which an extended working channel (EWC), which may be a smart extended working channel (sEWC) including an electromagnetic (EM) sensor, is advanced to permit access of a catheter to the luminal network of the patient. As compared to an EWC, the sEWC includes an EM sensor disposed on or adjacent to a distal end of the sEWC that is configured for use with an electromagnetic navigation (EMN) or tracking system, which tracks the location of EM sensors, such as for example, the EM sensor of the sEWC. The catheter includes a camera disposed on or adjacent to a distal end of the catheter that is configured to capture real-time images of the patient’s anatomy as the catheter is navigated through the luminal network of the patient. In this manner, the catheter is advanced through the sEWC and into the luminal network of the patient. It is envisioned that the catheter may be selectively locked to the sEWC to selectively inhibit, or permit, movement of the catheter relative to the sEWC. In embodiments, the catheter may include an EM sensor disposed on or adjacent to the distal end of the catheter. Although generally described as utilizing EM sensors, it is envisioned that the sEWC and/or the catheter may utilize any suitable sensor for detecting and/or identifying a position of the sEWC and/or catheter within the luminal network of the patient, such as for example, an inertial measurement unit (IMU).

The surgical system generates a 3-dimensional (3D) representation of the airways of the patient using pre-procedure images, such as for example, CT, CBCT, or MRI images and identifies anatomical landmarks within the 3D representation, such as for example, bifurcations or lesions. During a registration process of a surgical procedure, a location of the EM sensor of the sEWC is periodically identified and stored as a data point as the sEWC and catheter are navigated through the luminal network of the patient. As can be appreciated, the registration process may require the sEWC and catheter to be navigated within and survey particular portions of the patient’s luminal network, such as for example, the right upper lobe, the left upper lobe, the right lower lobe, the left lower lobe, and the right middle lobe. In embodiments this registration step may be considered a first estimated registration of the EM sensor to the patient’s anatomy, which may be used in addition to further data to increase and/or more robustly register the EM sensor to the patient’s anatomy as compared to utilizing a single registration step.

The working channel of the sEWC defines an aperture at a distal end thereof that includes an inner dimension that is less than an inner dimension of the working channel. The working channel of the sEWC includes a transition portion that includes an inner dimension that decreases from the inner dimension of the working channel to the inner dimension of the aperture in a proximal to distal direction. In this manner, the transition portion abuts a distal end portion of the catheter to inhibit or otherwise prevent the catheter from passing entirely through the working channel. Although generally described as abutting the transition portion of the sEWC, it is envisioned that the catheter may be inhibited from extending distal of the distal end portion of the sEWC using any suitable means located as any suitable location, such as for example, at a proximal end portion of the sEWC or catheter. The transition portion includes a pattern disposed or defined thereon to identify or otherwise determine a rotational and/or longitudinal position of the distal end portion of the catheter with respect to the sEWC, such as for example, the EM sensor. It is envisioned that the pattern 86 may be any suitable pattern configured to encode data, such as for example, one dimensional barcodes, DataMatrix, Maxicode, PDF417, QR Code^{®}, three-dimensional barcodes, and PM code. The rotational and longitudinal positions encoded in the pattern 86 reference a position of the EM sensor 72 of the sEWC 70.

It is envisioned that the surgical system may synthesize or otherwise generate virtual images from the 3D representation at various camera poses in proximity to the estimated location of the EM sensor within the airways of the patient. In this manner, a location within the 3D representation corresponding to the location data obtained from the EM sensors can be identified. The system generates virtual 2D or 3D images from the 3D representation corresponding to different perspectives or poses of the virtual camera viewing the patient’s airways within the 3D representation. The real-time images captured by the camera are compared to the generated 2D or 3D virtual images and the virtual 2D or 3D image having a perspective or pose that most closely approximates the perspective or pose of the camera is identified. In this manner, the location of the identified virtual image within the 3D representation is correlated to the location of the EM sensors of the sEWC and/or the rotational and/or longitudinal position of the distal end of the catheter relative to the sEWC, and a pose of the sEWC within the patient’s luminal network can be determined in six degrees-of-freedom. Although generally described as using anatomical landmarks to estimate a position of the second catheter within the patient’s luminal network, it is envisioned that any suitable method of estimating a position of the catheter and/or sEWC utilizing images captured by a camera may be utilized without departing from the scope of the present disclosure.

These and other aspects of the disclosure will be described in further detail hereinbelow. Although generally described with reference to the lung, it is contemplated that the systems and methods described herein may be used with any structure within the patient’s body, such as the liver, kidney, prostate, gynecological, amongst others.

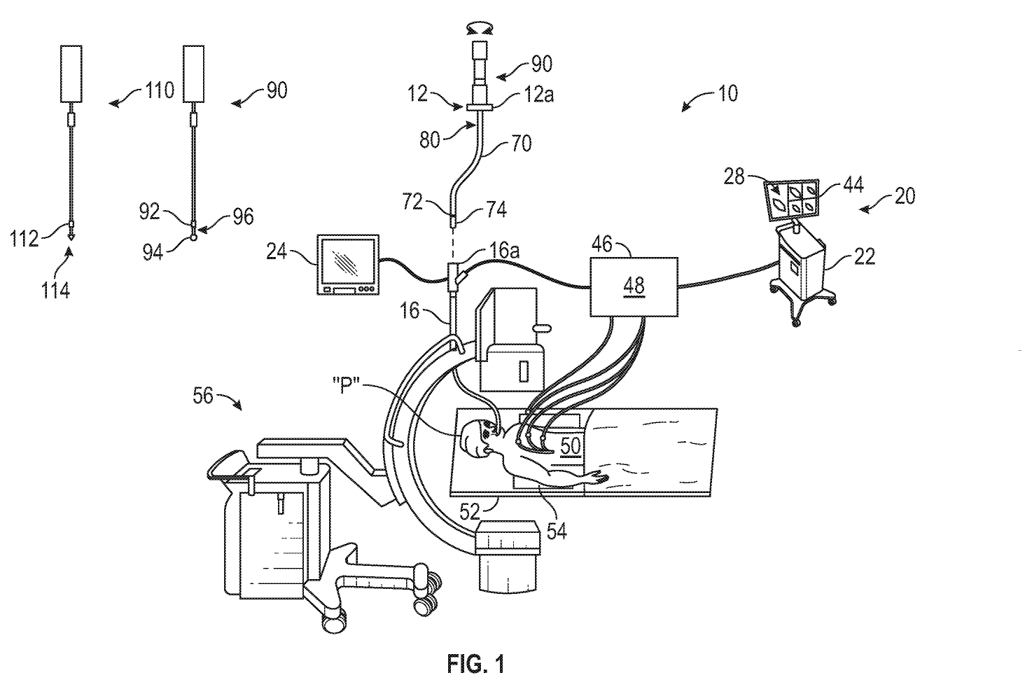

Turning now to the drawings, FIG. 1 illustrates a system 10 in accordance with the disclosure facilitating navigation of a medical device through a luminal network and to an area of interest. As will be described in further detail hereinbelow, the surgical system 10 is generally configured to identify target tissue, automatically register real-time images captured by a surgical instrument to a generated 3-dimensional (3D) model, and navigate the surgical instrument to the target tissue.

The system 10 includes a catheter guide assembly 12 including a first catheter, which may be any suitable catheter and in embodiments, may be an extended working channel (EWC) 70, which may be a smart extended working channel (sEWC) including an electromagnetic (EM) sensor. In one embodiment, the sEWC 70 is inserted into a bronchoscope 16 for access to a luminal network of the patient P. In this manner, the sEWC 70 may be inserted into a working channel of the bronchoscope 16 for navigation through a patient P’s luminal network, such as for example, the lungs. It is envisioned that the sEWC 70 may itself include imaging capabilities via an integrated camera or optics component (not shown) and therefore, a separate bronchoscope 16 is not strictly required. In embodiments, the sEWC 70 may be selectively locked to the bronchoscope 16 using a bronchoscope adapter 16a. In this manner, the bronchoscope adapter 16a is configured to permit motion of the sEWC 70 relative to the bronchoscope 16 (which may be referred to as an unlocked state of the bronchoscope adapter 16a) or inhibit motion of the sEWC 14 relative to the bronchoscope 16 (which may be referred to as a locked state of the bronchoscope adapter 16a). Bronchoscope adapters 16a are currently marketed and sold by Medtronic PLC under the brand names EDGE^{®} Bronchoscope Adapter or the ILLUMISITE^{®} Bronchoscope Adapter, and are contemplated as being usable with the disclosure.

As compared to an EWC, the sEWC 70 may include one or more EM sensors 72 disposed in or on the sEWC 70 at a predetermined distance from the distal end 74 of the sEWC 70. It is contemplated that the EM sensor 72 may be a five degree-of-freedom sensor or a six degree-of-freedom sensor. As can be appreciated, the position and orientation of the EM sensor 72 of the sEWC 70 relative to a reference coordinate system, and thus a distal portion of the sEWC 70 within an electromagnetic field can be derived. Catheter guide assemblies 12 are currently marketed and sold by Medtronic PLC under the brand names SUPERDIMENSION^{®} Procedure Kits, ILLUMISITE^{TM} Endobronchial Procedure Kit, ILLUMISITE^{TM} Navigation Catheters, or EDGE^{®} Procedure Kits, and are contemplated as being usable with the disclosure.

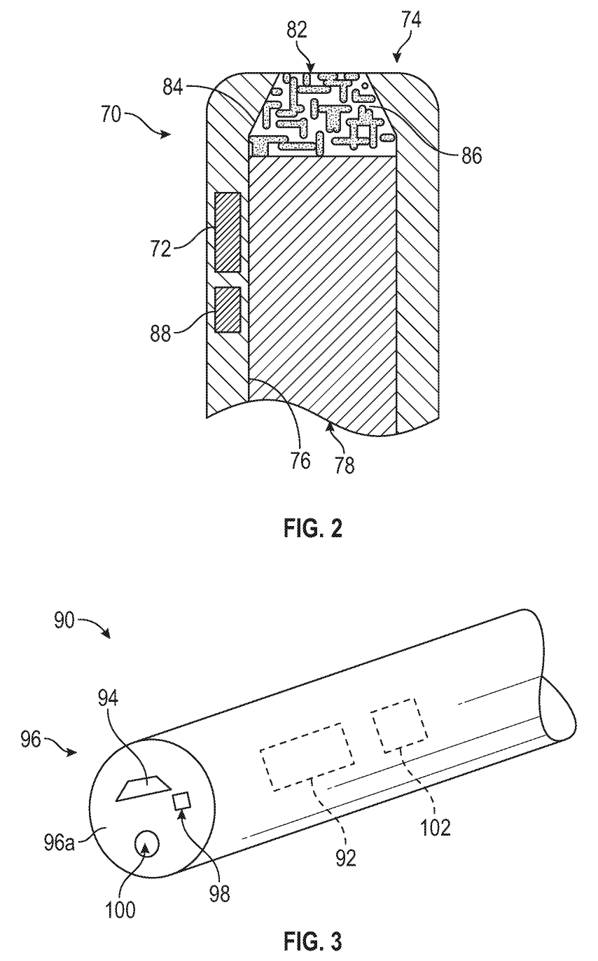

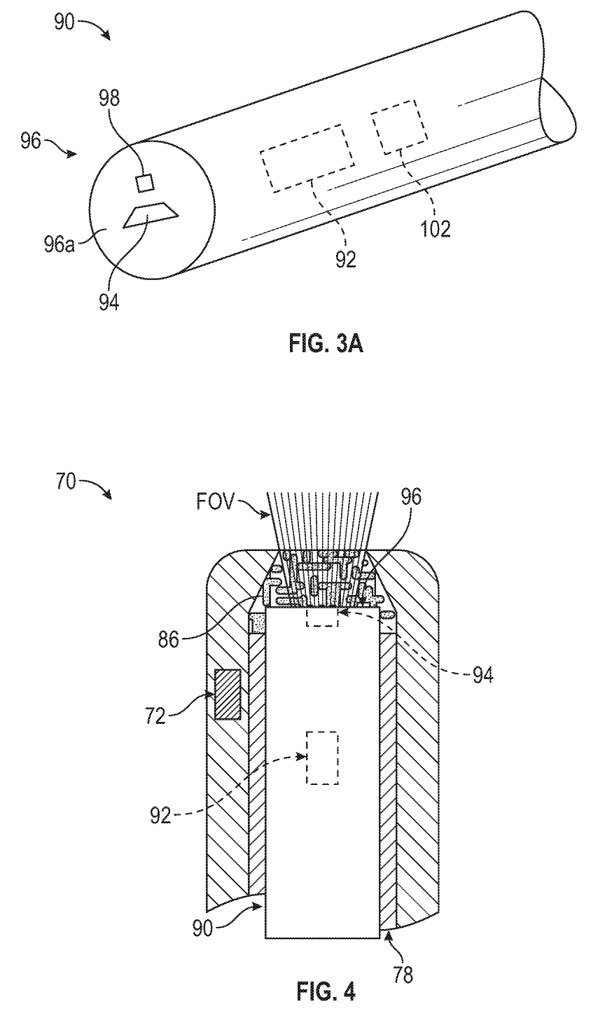

Continuing with FIG. 1 and with additional reference to FIG. 2, the sEWC 70 includes an inner surface 76 defining a working channel 78 extending through a proximal end 80 of the sEWC 70 and the distal end 74. The working channel 78 defines an aperture 82 adjacent to and extending through the distal end 74 and including an inner dimension that is less than an inner dimension of the working channel 78. In this manner, the inner surface 76 of the working channel 78 defines a transition portion 84 where the inner dimension of the working channel 78 transitions to the smaller inner dimension of the aperture 82. Although generally illustrated as defining a conical frustrum or tapered profile, it is envisioned that the transition portion 84 of the working channel 76 may define any suitable profile depending upon the design needs of the system 10, such as for example, concave, convex, stepped, and curvilinear without departing from the scope of the disclosure. As will be described in further detail hereinbelow, the inner dimension of the aperture 82 defines an inner dimension that is less than an outer dimension of a second medical device, which in embodiments, may be a camera catheter 90 (FIG. 3). In this manner, as the camera catheter 90 is advanced within the working channel 76 of the sEWC 70 towards the distal end 74, a distal end 96 of the camera catheter 90 abuts or otherwise contacts the transition portion 84 and inhibits the camera catheter 90 from extending through the aperture 82. Although generally described as abutting the transition portion 84 of the sEWC 70, it is envisioned that the camera catheter 90 may be inhibited from extending distal of the distal end 74 of the sEWC 70 using any suitable means located as any suitable location, such as for example, at a proximal end portion of the sEWC 70 or camera catheter 90.

Continuing with FIG. 2, a pattern 86 is disposed on the transition portion 84 of the working channel 78. The pattern 86 encodes or otherwise defines rotational positions about a circumference of the sEWC 70 and a longitudinal distance relative to the distal end 74 of the sEWC 70. It is envisioned that the pattern 86 may be any suitable pattern configured to encode data, such as for example, one dimensional barcodes, DataMatrix, Maxicode, PDF417, QR Code^{®}, three-dimensional barcodes, and PM code. The rotational and longitudinal positions, such as for example, the pose, encoded in the pattern 86 reference a position of the EM sensor 72 of the sEWC 70. As can be appreciated, referencing the position of the EM sensor 72 enables the system to identify a position of the distal end 96 of the camera catheter 90 in six degrees of freedom. It is envisioned that the pattern 86 may be disposed on the transition portion 84 using any suitable means, such as for example a separate component coupled to the transition portion 84 (such as for example, an adhesive backed label, painting, dying, 3D printing, and 2D printing). It is also envisioned that the pattern 86 may be integrally formed within the transition portion 84 (such as for example, by etching and machining).

With reference to FIG. 3, the camera catheter 90 includes one or more EM sensors 92 and is configured to be inserted into the sEWC 70 and selectively locked into position relative to the sEWC 70. As can be appreciated, the EM sensor 92 disposed on the camera catheter 90 is separate from the EM sensor 72 disposed on the sEWC 70. As described hereinabove, the distal end 96 of the camera catheter 90 is configured to abut or otherwise contact a portion of the transition portion 84 of the sEWC 70, inhibiting further distal insertion or translation of the camera catheter 90 relative to the sEWC 70. In this manner, the outer dimension of the camera catheter 90 is larger than at least a portion of the inner dimension of the transition portion 84 of the sEWC 70. In embodiments, the EM sensor 92 of the camera catheter 90 may be disposed on or in the camera catheter 90 a predetermined proximal distance from the distal end portion 96 of the camera catheter 90. In this manner, the system 10 is able to determine a position of the distal end 96 of the camera catheter 90 within the luminal network of the patient P or relative to the distal end 74 of the sEWC 70. It is envisioned that the camera catheter 90 may be selectively locked relative to the sEWC 70 at any time, regardless of the position of the distal end 96 of the camera catheter 90 relative to the sEWC 70. It is contemplated that the camera catheter 90 may be selectively locked to a handle 12a of the catheter guide assembly 12 using any suitable means, such as for example, a snap fit, a press fit, a friction fit, a cam, one or more detents, threadable engagement, or a chuck clamp. It is envisioned that the EM sensor 92 of the camera catheter 90 may be a five degree-of-freedom sensor or a six degree-of-freedom sensor. As will be described in further detail hereinbelow, the position and orientation of the EM sensor 92 of the camera catheter 90 relative to a reference coordinate system, and thus the distal end 96 of the camera catheter 90, within an electromagnetic field can be derived.

At least one camera 94 is disposed on or adjacent to a distal end surface 96a of the camera catheter 90 and is configured to capture, for example, still images, real-time images, or real-time video. In embodiments, the camera catheter 90 may include one or more light sources 98 disposed on or adjacent to the distal end surface 96a of the camera catheter 90 or any other suitable location (such as for example, a side surface or a protuberance). The light source 98 may be or include, for example, a light emitting diode (LED), an optical fiber connected to a light source that is located external to the patient P, or combinations thereof, and may emit one or more of white, IR, or near infrared (NIR) light. In this manner, the camera 94 may be, for example, a white light camera, an IR camera, an NIR camera, a camera that is capable of capturing white light and NIR light, or combinations thereof. In one non-limiting embodiment, the camera 94 is a white light mini complementary metal-oxide semiconductor (CMOS) camera, although it is contemplated that the camera 94 may be any suitable camera, such as for example, a charge-coupled device (CCD), a CMOS, a N-type metal-oxide-semiconductor (NMOS), and in embodiments, may be an IR camera, depending upon the design needs of the system 10. In embodiments, the camera 94 may be a dual lens camera or a Red Blue Green and Depth (RGB-D) camera configured to identify a distance between the camera 94 and anatomical features within the patient P’s anatomy without departing from the scope of the disclosure. As described hereinabove, it is envisioned that the camera 94 may be disposed on the camera catheter 90, the sEWC 70, or the bronchoscope 16.

In embodiments, the camera catheter 90 may include a working channel 100 defined through a proximal portion (not shown) and the distal end surface 96a, although in embodiments, it is contemplated that the working channel 100 may extend through a sidewall of the camera catheter 90 depending upon the design needs of the camera catheter 90. As can be appreciated, the working channel 100 is configured to receive a locatable guide (not shown) or a surgical tool, such as for example, a biopsy tool 110 (FIG. 1). Although generally described as having a working channel, it is envisioned that the camera catheter 90 may not have a working channel, and rather, may include only the camera 94 and in some embodiments, the light source 98 (FIG. 3A).

Returning to FIG. 1, the system 10 generally includes an operating table 52 configured to support a patient P and monitoring equipment 24 coupled to the sEWC 70, the bronchoscope 16, or the second catheter endoscope 90 (e.g., for example, a video display for displaying the video images received from the video imaging system of the bronchoscope 12 or the camera 94 of the camera catheter 90), a locating or tracking system 46 including a tracking module 48, a plurality of reference sensors 50 and a transmitter mat 54 including a plurality of incorporated markers, and a workstation 20 having a computing device 22 including software and/or hardware used to facilitate identification of a target, pathway planning to the target, navigation of a medical device to the target, and/or confirmation and or determination of placement of, for example, the sEWC 70, the bronchoscope 16, the camera catheter 90, or a surgical tool, relative to the target.

The tracking system 46 is, for example, a six degrees-of-freedom electromagnetic locating or tracking system, or other suitable system for determining position and orientation of, for example, a distal portion the sEWC 70, the bronchoscope 16, the camera catheter 90, or a surgical tool 110, for performing registration of a detected position of one or more of the EM sensors 72 or 92 and a three-dimensional (3D) model generated from a CT, CBCT, or MRI image scan. The tracking system 46 is configured for use with the sEWC 70 and the camera catheter 90, and particularly with the EM sensors 72 and 92.

Continuing with FIG. 1, the transmitter mat 54 is positioned beneath the patient P. The transmitter mat 54 generates an electromagnetic field around at least a portion of the patient P within which the position of the plurality of reference sensors 50 and the EM sensors 72 and 92 can be determined with the use of the tracking module 48. In one non-limiting embodiment, the transmitter mat 54 generates three or more electromagnetic fields. One or more of the reference sensors 50 are attached to the chest of the patient P. In embodiments, coordinates of the reference sensors 50 within the electromagnetic field generated by the transmitter mat 54 are sent to the computing device 22 where they are used to calculate a patient coordinate frame of reference (e.g., for example, a reference coordinate frame). As will be described in further detail hereinbelow, registration is generally performed using coordinate locations of the 3D model and 2D images from the planning phase, with the patient P’s airways as observed through the bronchoscope 12 or camera catheter 90 and allow for the navigation phase to be undertaken with knowledge of the location of the EM sensors 72 and 92. It is envisioned that any one of the EM sensors 72 and 92 may be a single coil sensor that enables the system 10 to identify the position of the sEWC 70 or the camera catheter 90 within the EM field generated by the transmitter mat 54, although it is contemplated that the EM sensors 72 and 92 may be any suitable sensor and may be a sensor capable of enabling the system 10 to identify the position, orientation, and/or pose of the sEWC 70 or the camera catheter 90 within the EM field.

Although generally described with respect to EMN systems using EM sensors, the instant disclosure is not so limited and may be used in conjunction with flexible sensors, such as for example, fiber-bragg grating sensors, inertial measurement units (IMU), ultrasonic sensors, optical sensors, pose sensors (e.g., for example, ultra-wide band, global positioning system, fiber-bragg, radio-opaque markers), without sensors, or combinations thereof. In one non-limiting embodiment, in lieu of or in addition to the EM sensors 72 and 92, the sEWC 70 may include an IMU 88 and/or the camera catheter 90 may include an IMU 102. It is contemplated that the devices and systems described herein may be used in conjunction with robotic systems such that robotic actuators drive the sEWC 70 or bronchoscope 16 proximate the target.

In accordance with aspects of the disclosure, the visualization of intra-body navigation of a medical device (e.g., for example a biopsy tool or a therapy tool), towards a target (e.g., for example, a lesion) may be a portion of a larger workflow of a navigation system. An imaging device 56 (e.g., for example, a CT imaging device, such as for example, a cone-beam computed tomography (CBCT) device, including but not limited to Medtronic plc’s O-arm™ system) capable of acquiring 2D and 3D images or video of the patient P is also included in the particular aspect of system 10. The images, sequence of images, or video captured by the imaging device 56 may be stored within the imaging device 56 or transmitted to the computing device 22 for storage, processing, and display. In embodiments, the imaging device 56 may move relative to the patient P so that images may be acquired from different angles or perspectives relative to the patient P to create a sequence of images, such as for example, a fluoroscopic video. The pose of the imaging device 56 relative to the patient P while capturing the images may be estimated via markers incorporated with the transmitter mat 54. The markers are positioned under the patient P, between the patient P and the operating table 52, and between the patient P and a radiation source or a sensing unit of the imaging device 56. The markers incorporated with the transmitter mat 54 may be two separate elements which may be coupled in a fixed manner or alternatively may be manufactured as a single unit. It is contemplated that the imaging device 56 may include a single imaging device or more than one imaging device.

Continuing with FIG. 1 and with additional reference to FIG. 3, the workstation 20 includes a computer 22 and a display 24 that is configured to display one or more user interfaces 26 and/or 28. The workstation 20 may be a desktop computer or a tower configuration with the display 24 or may be a laptop computer or other computing device. The workstation 20 includes a processor 30 which executes software stored in a memory 32. The memory 32 may store video or other imaging data captured by the bronchoscope 16 or camera catheter 90 or pre-procedure images from, for example, a computer -tomography (CT) scan, Positron Emission Tomography (PET), Magnetic Resonance Imaging (MRI), Cone-beam CT, amongst others. In addition, the memory 32 may store one or more software applications 34 to be executed on the processor 30. Though not explicitly illustrated, the display 24 may be incorporated into a head mounted display such as an augmented reality (AR) headset such as the HoloLens offered by Microsoft Corp.

A network interface 36 enables the workstation 20 to communicate with a variety of other devices and systems via the Internet. The network interface 36 may connect the workstation 20 to the Internet via a wired or wireless connection. Additionally, or alternatively, the communication may be via an ad-hoc Bluetooth^{®} or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further image data and videos may be stored. The cloud storage system 38 may be remote from or on the premises of the hospital such as in a control or hospital information technology room. An input module 40 receives inputs from an input device such as a keyboard, a mouse, voice commands, amongst others. An output module 42 connects the processor 30 and the memory 32 to a variety of output devices such as the display 24. In embodiments, the workstation 20 may include its own display 44, which may be a touchscreen display.

In a planning or pre-procedure phase, the software application utilizes pre-procedure CT image data, either stored in the memory 32 or retrieved via the network interface 36, for generating and viewing a 3D model of the patient’s anatomy, enabling the identification of target tissue TT on the 3D model (automatically, semi-automatically, or manually), and in embodiments, allowing for the selection of a pathway through the patient’s anatomy to the target tissue. Examples of such an application is the ILOGIC^{®} planning and navigation suites and the ILLUMISITE^{®} planning and navigation suites currently marketed by Medtronic PLC. The 3D model may be displayed on the display 24 or another suitable display associated with the workstation 20, such as for example, the display 44, or in any other suitable fashion. Using the workstation 20, various views of the 3D model may be provided and/or the 3D model may be manipulated to facilitate identification of target tissue on the 3D model and/or selection of a suitable pathway to the target tissue.

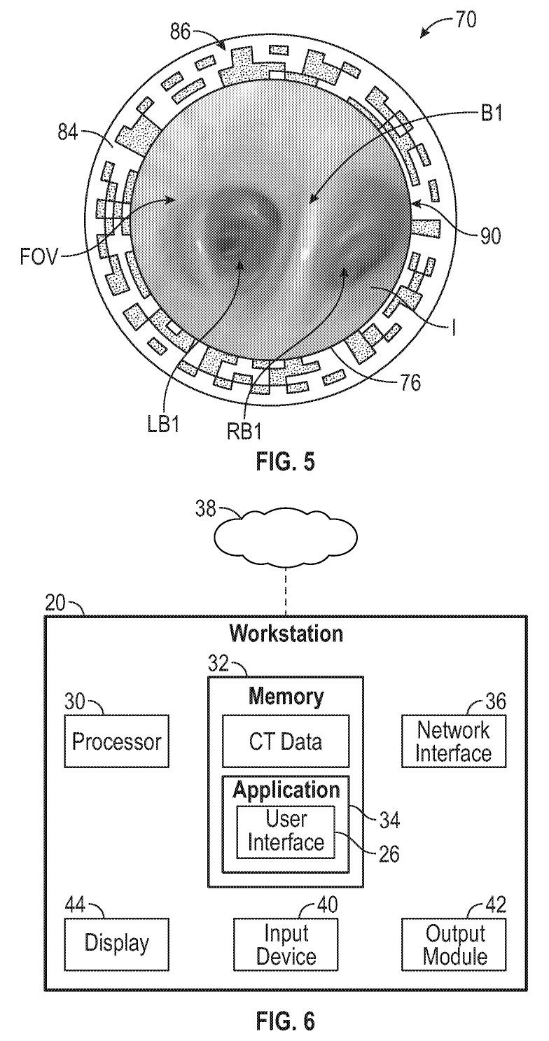

It is envisioned that the 3D model may be generated by segmenting and reconstructing the airways of the patient P’s lungs to generate a 3D airway tree. The reconstructed 3D airway tree includes various branches and bifurcations which, in embodiments, may be labeled using, for example, well accepted nomenclature such as RB1 (right branch 1), LB1 (left branch 1), or B1 (bifurcation one) (FIG. 5). In embodiments, the segmentation and labeling of the airways of the patient’s lungs is performed to a resolution that includes terminal bronchioles having a diameter of approximately less than 1 mm. As can be appreciated, segmenting the airways of the patient P’s lungs to terminal bronchioles improves the accuracy registration between the position of the sEWC 70 and camera catheter 90 and the 3D model, improves the accuracy of the pathway to the target, and improves the ability of the software application to identify the location of the sEWC 70 and camera catheter 90 within the airways and navigate the sEWC 70 and camera catheter 90 to the target tissue. Those of skill in the art will recognize that a variety of different algorithms may be employed to segment the CT image data set, including, for example, connected component, region growing, thresholding, clustering, watershed segmentation, or edge detection. It is envisioned that the entire reconstructed 3D airway tree may be labeled, or only branches or branch points within the reconstructed 3D airway tree that are located adjacent to the pathway to the target tissue.

In embodiments, the software stored in the memory 32 may identify and segment out a targeted critical structure within the 3D model. It is envisioned that the segmentation process may be performed automatically, manually, or a combination of both. The segmentation process isolates the targeted critical structure from the surrounding tissue in the 3D model and identifies its position within the 3D model. In embodiments, the software application segments the CT images to terminal bronchioles that are less than 1 mm in diameter such that branches and/or bifurcations are identified and labeled deep into the patient’s luminal network. As an be appreciated, this position can be updated depending upon the view selected on the display 24 such that the view of the segmented targeted critical structure may approximate a view captured by the camera 94 of the camera catheter 90.

As can be appreciated, the 3D model generated from previously acquired images may not provide a basis sufficient for accurate registration or guidance of medical devices or tools to a target during a navigation phase of the surgical procedure. In some cases, the inaccuracy is caused by deformation of the patient’s lungs during the surgical procedure relative to the lungs at the time of the acquisition of the previously acquired images. This deformation (CT-to-Body divergence) may be caused by many different factors including, for example, changes in the patient P’s body when transitioning from between a sedated state and a non-sedated state, the bronchoscope 16, the sEWC 70, or the camera catheter 90 changing the patient P’s pose, the bronchoscope 16, the sEWC 70, or the catheter 90 pushing the tissue, different lung volumes (e.g., for example, the previously acquired images are acquired during an inhale while navigation is performed as the patient P is breathing), different beds, a time period between when the previous images were captured and when the surgical procedure is being performed, a change in the lung shape due to, for example, a change in temperature or time of day between when the previous images were captured and when the surgical procedure is being performed, the effects of gravity on the patient P’s lungs due to the length of time the patient P is laying on the operating table 52, or diseases that were not present or have progressed since the time when the previous images were captured.

With additional reference to FIGS. 3 and 4, registration of the patient P’s location on the transmitter mat 54 may be performed by moving the EM sensors 72 and/or 92 through the airways of the patient P. In this manner, the software stored on the memory 32 periodically determines the location of the EM sensors 72 or 92 within the coordinate system as the sEWC 70 and/or the camera catheter 90 is moving through the airways using the transmitter mat 54, the reference sensors 50, and the tracking system 46. The location data may be represented on the user interface 26 as a marker or other suitable visual indicator, a plurality of which develop a point cloud having a shape that may approximate the interior geometry of the 3D model. The shape resulting from this location data is compared to an interior geometry of passages of a 3D model, and a location correlation between the shape and the 3D model based on the comparison is determined. In addition, the software identifies non-tissue space (e.g., for example, air filled cavities) in the 3D model. The software aligns, or registers, an image representing a location of the EM sensors 72 or 92 with the 3D model and/or 2D images generated from the 3D model, which are based on the recorded location data and an assumption that the sEWC 70 or the camera catheter 90 remains located in non-tissue space in a patient’s airways. In embodiments, a manual registration technique may be employed by navigating the sEWC 70 or second catheter 72 with the EM sensors 72 and 92 to pre-specified locations in the lungs of the patient P, and manually correlating the images from the bronchoscope 16 or the camera catheter 90 to the model data of the 3D model. Although generally described herein as utilizing a point cloud (e.g., for example, a plurality of location data points), it is envisioned that registration can be completed utilizing any number of location data points, and in one non-limiting embodiment, may utilize only a single location data point. It is envisioned that registration of the EM sensors 72 and/or 92 may be a first estimated registration that may be utilized in addition to additional data to increase the accuracy or robustness of the registration of the EM sensors 72 and/or 92 to the patient P’s anatomy as compared to utilizing a single registration step, as will be described in further detail hereinbelow.

Turning to FIGS. 3-6, during registration, and in embodiments, during navigation, CT-to-body divergence may be mitigated by integrating real-time images captured by the camera 94 of the camera catheter 90 as the sEWC 70, with the camera catheter 90 coupled thereto, is moving through the patient P’s airways. In this manner, the software stored on the memory 32 analyzes the pre-segmented pre-procedure CT model and identifies locations of anatomical landmarks, such as for example, bifurcations, airway walls, or lesions, although it is envisioned that the system 10 may utilize any suitable method of analyzing real-time images to identify and/or determine the position of the camera 94 within the patient P’s anatomy without departing from the scope of the present disclosure. In embodiments where anatomical landmarks are identified, the anatomical landmark may be a bifurcation, labeled as B1 in the user interface 26 (FIG. 5). As the sEWC 70 and camera catheter 90 are moving through the airways, images I of the patient P’s anatomy are captured using the camera 94 in real-time from a perspective of looking out from the distal end 96 of the camera catheter 90. The real-time images I captured by camera 94 are continuously segmented via the software stored on the memory 32 to identify anatomical landmarks within the real-time images I (FIG. 5). The software stored on the memory 32 continuously analyzes the captured images I in real-time and identifies commonalities between the anatomical landmarks identified by the software application in the real-time images I and the pre-procedure images, illustrated as bifurcation B1 in FIG. 5. A distance between the camera 94 and the anatomical landmarks identified in the real-time images I is determined using, for example, the EM sensors 72 or 92, the predetermined distance between the EM sensors 72 or 92, a known zoom level of the real-time images I captured by the camera 94, and the pattern 86 disposed on the transition portion 84 of the camera catheter 90, although it is contemplated that the distance between the camera 94 and the identified anatomical landmarks may be determined using any suitable means without departing from the scope of the present disclosure, such as for example, data obtained from a dual lens camera or RGB-D camera. Using the distance between the camera 94 and the anatomical landmark, the location of the sEWC 70 and/or the camera catheter 90 within the coordinate system is recorded and utilized in addition to the location data obtained by the EM sensors 72 and 92 to register a location of the sEWC 70 or the camera catheter 90 to the 3D model. In embodiments where a dual lens camera of RGB-D camera is utilized, a determined distance between the camera 94 and the identified anatomical landmark is determined, adding redundancy and increasing the accuracy of the distance determination. It is contemplated that data points where distance determined using the camera 94 correlates to the distance determined using the EM sensors 72 and/or 92 may be weighted or otherwise afforded greater importance during registration.

With continued reference to FIGS. 3-6, as described hereinabove, the sEWC 70 may be constructed without a camera, enabling the outer dimension of the sEWC 70 to become smaller and enabling the sEWC to navigate further into the luminal network of the patient P or otherwise advance into airways having a smaller diameter as compared to an sEWC having a camera. As can be appreciated, although an sEWC 70 constructed without a camera enables an outer dimension of the sEWC 70 to become smaller, the camera 94 of the camera catheter 90 may rotate or otherwise translate relative to the distal end 74 of the sEWC 70 as the sEWC 70, with the camera catheter 90 locked thereto, as the sEWC 70 and the camera catheter 90 are navigated within the luminal network of the patient. Although a proximal end portion of the camera catheter 90 is locked relative to the sEWC 70, it is possible that the camera catheter 90 may twist or otherwise move relative to the sEWC 70, and such movement may increase along the length of the camera catheter 90 in a proximal to distal direction. Further, as the camera catheter 90 is released or otherwise unlocked from the sEWC 70, the rotational and longitudinal position of the distal end 96 may shift relative to the distal end 74 of the sEWC, leading to inaccuracies when positioning a surgical tool, such as a biopsy tool 110, relative to the target tissue.

To account for or otherwise solve positional and rotational discrepancies between the distal end 74 of the sEWC 70 and the camera catheter 90, the system 10 utilizes the pattern 86 disposed on the transition portion 84 of the sEWC 70 to identify or otherwise determine a rotational and/or longitudinal position of the distal end 96 of the camera catheter 90 with respect to the EM sensor 72 of the sEWC 70 and vice-versa. In this manner, when the distal end 96 of the camera catheter 90 is advanced within the working channel 78 of the sEWC 70, and the distal end 96 is inhibited from extending past the distal end 74 of the sEWC 70 by abutting otherwise contacting a portion of the transition portion 84 (FIG. 4), and the pattern 86 is visible within a Field of View (FOV) of the camera 94 of the camera catheter 90 (FIG. 5) in addition to the patient P’s anatomy distal of the distal end 74 of the sEWC 70. As described hereinabove, the pattern 86 is encoded with or otherwise defines rotational positions about a circumference of the sEWC 70 and a longitudinal distance relative to the distal end 74 of the sEWC 70. The rotational and longitudinal positions encoded in the pattern 86 reference a position of the EM sensor 72 of the sEWC 70. In this manner, the software stored in the memory 32 analyzes the images I captured by the camera 94 and identifies or otherwise determines a position of the distal end 96 of the camera catheter 90 relative to the EM sensor 72 of the sEWC 70 in six degrees-of-freedom (e.g., for example, registers the camera 94 to the EM sensor 72). The registration of the camera 94 to the patient P’s anatomy is translated from camera 94 to patient P anatomy coordinates to EM sensor 72 to patient P anatomy coordinates by correlating the estimated registration of the camera 94 to the patient P’s airways to the estimated registration of the camera 94 to the EM sensor 72. In this manner, a second estimated registration of the EM sensor 94 to the patient P’s anatomy is generated. As can be appreciated, utilizing or otherwise combining the first estimated registration of the EM sensor 72 to patient P anatomy and the second estimated registration of the EM sensor 72 to patient P anatomy more accurately or more robustly registers the EM sensor 72 to patient P anatomy as compared to utilizing a single registration step. It is envisioned that the software stored in the memory 32 may continuously analyze the real-time images I captured by the camera 94 and update the position of the camera catheter 90 in six degrees-of-freedom in real-time. In embodiments, the real-time position of the camera catheter 90 may be used as an additional data point for localizing the position of the camera catheter 90 and/or the sEWC 70 within the patient P’s airways, may attach or otherwise assign image orientation information to the real-time images (such as for example, a location of the patient P’s head, feet, left hand, right hand, back, and front), which may be displayed on the user interface 26 or stored in the memory 32 to enhance the user experience, or may be utilized by the software stored in the memory 32 to align or otherwise register virtual navigation views to the real-time images captured by the camera 94.

In embodiments, the software stored on the memory 32 correlates the determined position of the anatomical landmarks identified in the images I captured by the camera 94 of the camera catheter 90 with the identified positional information of the distal end 96 of the camera catheter 90 relative to the EM sensor 72 of the sEWC 70. In this manner, the position and orientation, or the pose, of the distal end 74 of the sEWC 70 and/or the pose of the distal end 96 of the camera catheter 90 may be identified in six degrees-of-freedom. With the position and orientation of the distal end 74 of the sEWC 70 and/or the distal end 96 of the camera catheter 90 identified in six degrees-of-freedom, the camera catheter 90 may be decoupled from the sEWC 70 and withdrawn from the working channel 78 of the sEWC 70 and the target tissue TT may be treated.

Returning to FIG. 1, registration of the patient P’s location on the transmitter mat 54 may be performed by moving the EM sensors 72 and/or 92 through the airways of the patient P. In this manner, the software stored on the memory 32 periodically determines the location of the EM sensors 72 or 92 within the coordinate system as the sEWC 70 and the camera catheter 90 is moving through the airways using the transmitter mat 54, the reference sensors 50, and the tracking system 46. The location data may be represented on the user interface 26 as a marker or other suitable visual indicator, a plurality of which develop a point cloud having a shape that may approximate the interior geometry of the 3D model. The shape resulting from this location data is compared to an interior geometry of passages of the 3D model, and a location correlation between the shape and the 3D model based on the comparison is determined. In addition, the software identifies non-tissue space (e.g., for example, air filled cavities) in the 3D model. The software aligns, or registers, an image representing a location of the EM sensors 72 or 92 with the 3D model and/or 2D images generated from the 3D model, which are based on the recorded location data and an assumption that the sEWC 70 or the camera catheter 90 remains located in non-tissue space in a patient P’s airways. In embodiments, a manual registration technique may be employed by navigating the sEWC 70 or the camera catheter 90 with the EM sensors 72 and 92 to pre-specified locations in the lungs of the patient P, and manually correlating the images from the bronchoscope 16 or the camera catheter 90 to the model data of the 3D model. Although generally described herein as utilizing a point cloud (e.g., for example, a plurality of location data points), it is envisioned that registration can be completed utilizing any number of location data points, and in one non-limiting embodiment, may utilize only a single location data point. As described hereinabove, the identified rotational and longitudinal position of the distal end 96 of the camera catheter 90 relative to the EM sensor 72 of the sEWC 70 may be utilized as an additional data point during registration to improve the accuracy of registration.

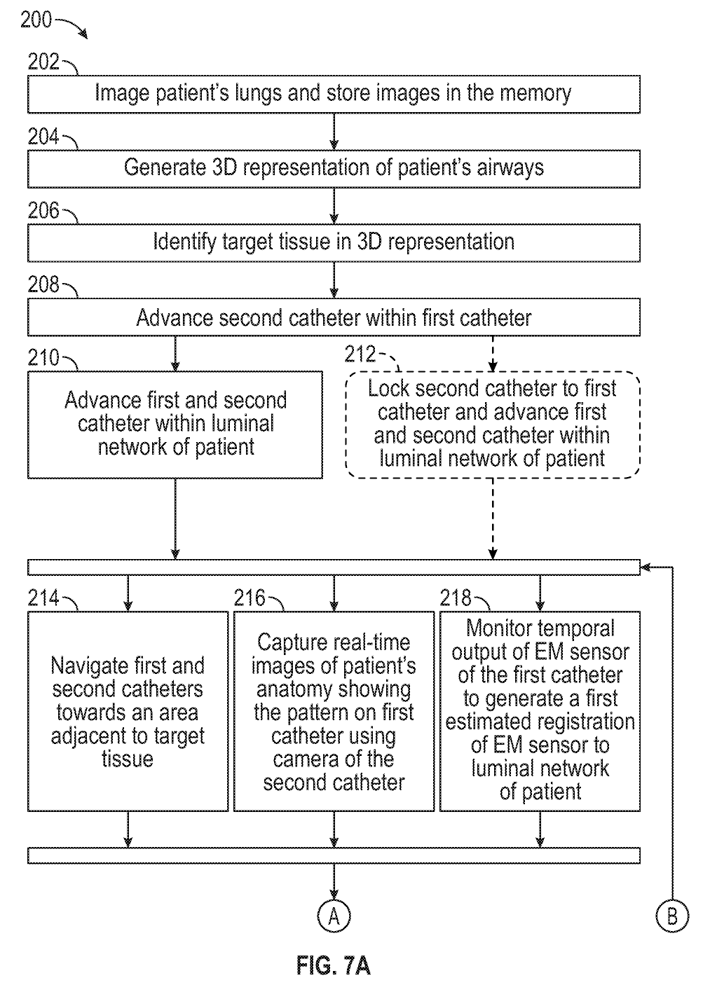

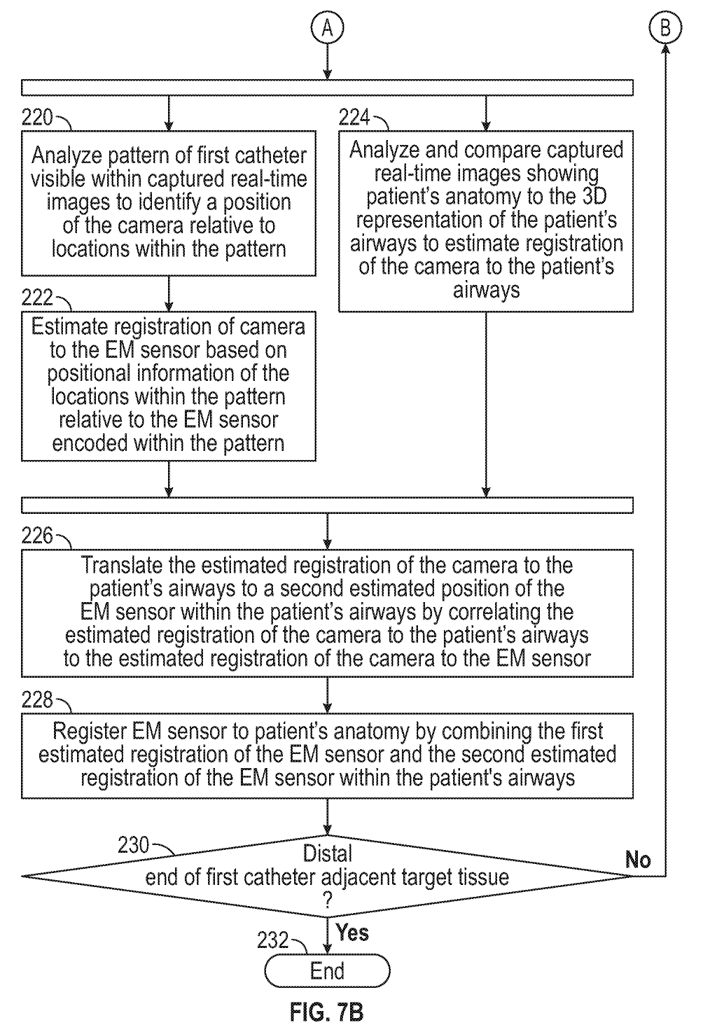

With reference to FIGS. 7A and 7B, a method of navigating a medical device within a luminal network of a patient is described and generally identified by reference numeral 200. Initially, in step 202, the patient P’s lungs are imaged using any suitable imaging modality (such as for example, CT, MRI, and CBCT) and the images are stored in the memory 32 associated with the workstation 20. As can be appreciated, imaging of the patient P’s lungs at step 202 may be performed at any suitable time, such as for example, pre-operatively, intra-operatively, or peri-operatively. In step 204, the images stored on the memory 32 are utilized to generate and view a 3D representation of the airways of the patient P’s lungs. Thereafter, an area of interest or target tissue are identified in the 3D representation in step 206. With the area of interest identified, in step 208, a second catheter is advanced within a first catheter until a distal end of the second catheter. In step 210, the first catheter, with the second catheter advanced therein, is advanced within the luminal network of the patient P’s lungs. Optionally, in step 212, the second catheter may be locked to the first catheter before advancing the first catheter and the second catheter within the luminal network of the patient P’s lungs. In step 214, the first and second catheters are navigated towards an area adjacent to the target tissue within the patient P’s lungs. In parallel with step 214, in step 216, real-time images of the patient P’s anatomy are captured by a camera of the second catheter as the first and second catheters are navigated within the luminal network of the patient P’s lungs with a pattern of the first catheter visible within the captured real-time images. In parallel with steps 214 and 216, in step 218, a temporal output of the EM sensor of the first catheter is monitored to generate a first estimated registration of the EM sensor to the luminal network of the patient P. In step 220, as the camera of the second catheter captures images I in real-time, the pattern of the first catheter that is visible within the FOV of the camera of the second catheter is analyzed in real-time to identify a position of the camera relative to locations within the pattern. In step 222, registration of the camera of the second catheter to the EM sensor of the first catheter is estimated based on positional information of the locations within the pattern relative to the EM sensor encoded within the pattern. In parallel with step 220, in step 224, the real-time images captured by the camera of the second catheter showing the patient’s anatomy are analyzed and compared to the 3D representation of the airways of the patient P to estimate registration of the camera to the patient P’s airways. In step 226, the estimated registration of the camera of the second catheter to the patient P’s airways is translated to a second estimated registration of the EM sensor of the first catheter within the patient P’s airways by correlating the estimated registration of the camera of the second catheter to the patient P’s airways to the estimated registration of the camera of the second catheter to the EM sensor of the first catheter. In step 228, the EM sensor is registered to the patient P’s anatomy by combining the first estimated registration of the EM sensor and the second estimated registration of the EM sensor within the patient P’s airways. In step 230, it is determined if the distal end of the first catheter is disposed adjacent to, or is disposed at a desired location relative to, the target tissue or area of interest. If it is determined that the distal end of the first catheter is not disposed adjacent to, or disposed at the desired location relative to, the target tissue or area of interest, the method returns to steps 214, 216, and 218. If it is determined that the distal end of the first catheter is disposed adjacent to, or disposed at the desired location relative to, the target tissue or area of interest, the method ends at step 232. As can be appreciated, the above-described method may be repeated as many times as necessary and may be performed both globally and locally within the airways of the patient P.

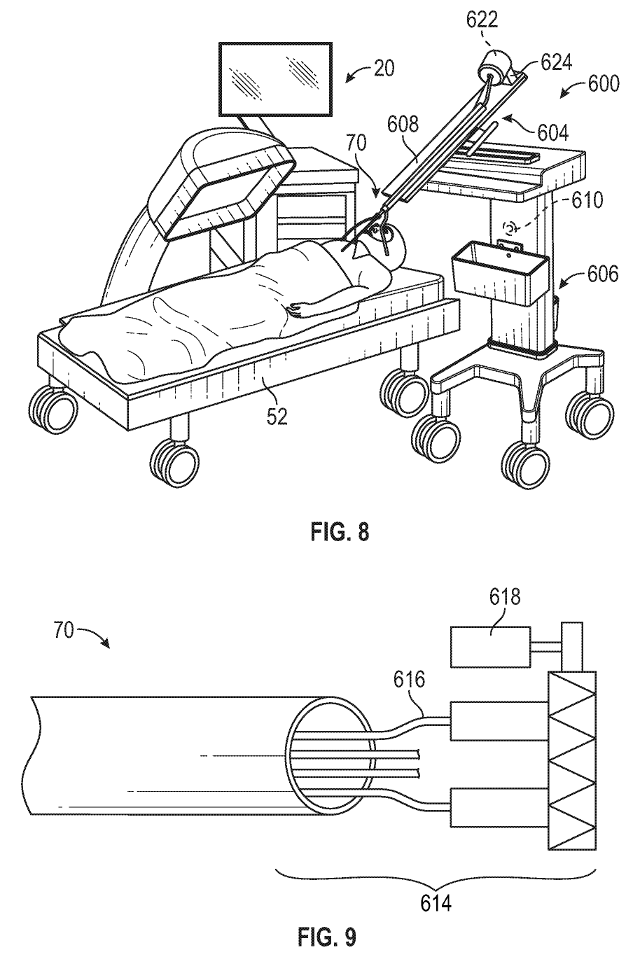

Turning to FIGS. 8 and 9, it is envisioned that the system 10 may include a robotic surgical system 600 having a drive mechanism 602 including a robotic arm 604 operably coupled to a base or cart 606, which may, in embodiments, be the workstation 20. The robotic arm 604 includes a cradle 608 that is configured to receive a portion of the sEWC 14. The sEWC 14 is coupled to the cradle 608 using any suitable means (e.g., for example, straps, mechanical fasteners, and/or couplings). It is envisioned that the robotic surgical system 600 may communicate with the sEWC 14 via electrical connection (e.g., for example, contacts and/or plugs) or may be in wireless communication with the sEWC 70 to control or otherwise effectuate movement of one or more motors (FIG. 8) disposed within the sEWC 70 and in embodiments, may receive images captured by a camera (not shown) associated with the sEWC 70. In this manner, it is contemplated that the robotic surgical system 600 may include a wireless communication system 610 operably coupled thereto such that the sEWC 70 may wirelessly communicate with the robotic surgical system 600 and/or the workstation 20 via Wi-Fi, Bluetooth^{®}, for example. As can be appreciated, the robotic surgical system 600 may omit the electrical contacts altogether and may communicate with the sEWC 70 wirelessly or may utilize both electrical contacts and wireless communication. The wireless communication system 610 is substantially similar to the network interface 36 (FIG. 6) described hereinabove, and therefore, will not be described in detail herein in the interest of brevity. As indicated hereinabove, the robotic surgical system 600 and the workstation 20 may be one in the same, or in embodiments, may be widely distributed over multiple locations within the operating room. It is contemplated that the workstation 20 may be disposed in a separate location and the display 44 (FIGS. 1 and 6) may be an overhead monitor disposed within the operating room.

As indicated hereinabove, it is envisioned that the sEWC 70 may be manually actuated via cables or push wires, or for example, may be electronically operated via one or more buttons, joysticks, toggles, actuators (not shown) operably coupled to a drive mechanism 614 disposed within an interior portion of the sEWC 70 that is operably coupled to a proximal portion of the sEWC 70, although it is envisioned that the drive mechanism 614 may be operably coupled to any portion of the sEWC 70. The drive mechanism 614 effectuates manipulation or articulation of the distal end of the sEWC 70 in four degrees of freedom or two planes of articulation (e.g., for example, left, right, up, or down), which is controlled by two push-pull wires, although it is contemplated that the drive mechanism 614 may include any suitable number of wires to effectuate movement or articulation of the distal end of the sEWC 70 in greater or fewer degrees of freedom without departing from the scope of the disclosure. It is contemplated that the distal end of the sEWC 70 may be manipulated in more than two planes of articulation, such as for example, in polar coordinates, or may maintain an angle of the distal end relative to the longitudinal axis of the sEWC 70 while altering the azimuth of the distal end of the sEWC 70 or vice versa. In one non-limiting embodiment, the system 10 may define a vector or trajectory of the distal end of the sEWC 70 in relation to the two planes of articulation.

It is envisioned that the drive mechanism 614 may be cable actuated using artificial tendons or pull wires 616 (e.g., for example, metallic, non-metallic, and/or composite) or may be a nitinol wire mechanism. In embodiments, the drive mechanism 614 may include motors 618 or other suitable devices capable of effectuating movement of the pull wires 616. In this manner, the motors 618 are disposed within the sEWC 70 such that rotation of an output shaft the motors 618 effectuates a corresponding articulation of the distal end of the sEWC 70.

Although generally described as having the motors 618 disposed within the sEWC 70, it is contemplated that the sEWC 70 may not include motors 618 disposed therein. Rather, the drive mechanism 614 disposed within the sEWC 14 may interface with motors 622 disposed within the cradle 608 of the robotic surgical system 600. In embodiments, the sEWC 70 may include a motor or motors 618 for controlling articulation of the distal end 74 of the sEWC 70 in one plane (e.g., for example, left/null or right/null) and the drive mechanism 624 of the robotic surgical system 600 may include at least one motor 622 to effectuate the second axis of rotation and for axial motion. In this manner, the motor 618 of the sEWC 70 and the motors 622 of the robotic surgical system 600 cooperate to effectuate four-way articulation of the distal end of the sEWC 70 and effectuate rotation of the sEWC 70. As can be appreciated, by removing the motors 618 from the sEWC 70, the sEWC 70 becomes increasingly cheaper to manufacture and may be a disposable unit. In embodiments, the sEWC 70 may be integrated into the robotic surgical system 600 (e.g., for example, one piece) and may not be a separate component.

From the foregoing and with reference to the various figures, those skilled in the art will appreciate that certain modifications can be made to the disclosure without departing from the scope of the disclosure.

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable and non-removable media implemented in any method or technology for storage of information such as for example, computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 20.

What is claimed is:

1. A system for performing a surgical procedure, comprising:
a first catheter, the first catheter including:
an inner surface defining a channel extending through a proximal end portion of the first catheter and a distal end portion of the first catheter;
an aperture disposed adjacent to the distal end portion of the first catheter, the aperture including an inner dimension that is less than an inner dimension of the channel, wherein the aperture is in open communication with the channel;
a transition portion disposed on the inner surface of the channel and adjacent to the aperture, wherein the transition portion includes an inner dimension that increases from the inner dimension of the aperture to the inner dimension of the channel in a distal to proximal direction; and
a pattern disposed on the transition portion, the pattern storing positional information correlating a location on the pattern to a location on the first catheter;
a second catheter receivable within the channel, the second catheter including a camera having a field of view encompassing the pattern of the first catheter; and
a workstation operably coupled to the first catheter and the second catheter, the workstation including a processor and a memory storing thereon instructions, which when executed by the processor cause the processor to:
receive images captured by the camera of the second catheter, the pattern being visible within the received images; and
analyze the pattern visible within the received images to determine a position of the second catheter relative to the first catheter.

2. The system according to claim 1, wherein the second catheter includes an outer dimension that is greater than the inner dimension of the aperture to inhibit distal advancement of the second catheter past the aperture.

3. The system according to claim 1, wherein the transition portion defines a tapered surface extending towards an inner portion of the channel.

4. The system according to claim 1, wherein the pattern is selected from the group consisting of a one-dimensional barcode, DataMatrix, Maxicode, PDF417, a QR Code, a three-dimensional barcode, and a PM code.

5. The system according to claim 1, wherein the positional information is a rotational position and a longitudinal position.

6. The system according to claim 1, wherein the first catheter includes a positional sensor, wherein the positional sensor is disposed a predetermined distance from the distal end portion of the first catheter.

7. The system according to claim 6, wherein the location on the first catheter is a location of the positional sensor.

8. The system according to claim 6, wherein the positional sensor is an electromagnetic sensor.

9. The system according to claim 6, wherein the positional sensor is an inertial measurement unit.

10. The system according to claim 1, wherein the pattern is etched into a surface of the transition portion.

11. A catheter, comprising:
a proximal end portion;
a distal end portion;
an inner surface, the inner surface defining a channel extending through the proximal end portion and the distal end portion;
an aperture disposed adjacent to the distal end portion, the aperture including an inner dimension that is less than an inner dimension of the channel, wherein the aperture is in open communication with the channel;
a transition portion disposed on the inner surface of the channel and adjacent to the aperture, wherein the transition portion includes an inner dimension that increases from the inner dimension of the aperture to the inner dimension of the channel in a distal to proximal direction; and
a pattern disposed on the transition portion, the pattern storing positional information correlating a location on the pattern to a location on the catheter.

12. The catheter according to claim 11, wherein the pattern is selected from the group consisting of a one-dimensional barcode, DataMatrix, Maxicode, PDF417, a QR Code, a three-dimensional barcode, and a PM code.

13. The system according to claim 11, wherein the positional information is a rotational position and a longitudinal position.

14. The system according to claim 11, further comprising a positional sensor, wherein the positional sensor is disposed a predetermined distance from the distal end portion.

15. The system according to claim 14, wherein the location on the catheter is a location of the positional sensor.

16. A method of navigating a medical device within a luminal network of a patient, comprising:
advancing a first catheter within a patient’s luminal network;
advancing a second catheter within a channel defined through the first catheter;
capturing images from a camera disposed on the second catheter, the captured images including a view of a pattern disposed on a portion of an inner surface of the channel of the first catheter, wherein the pattern stores positional information correlating a location on the pattern to a location on the first catheter; and
analyzing the pattern visible within the captured images to determine a position of the second catheter relative to the location on the first catheter.

17. The method according to claim 16, wherein analyzing the pattern visible within the captured images includes analyzing the pattern visible within the captured images to determine a rotational position of a distal portion of the second catheter and a longitudinal position of the distal portion of the second catheter relative to the location on the first catheter.

18. The method according to claim 16, wherein analyzing the pattern visible within the captured images includes analyzing the pattern visible within the captured images to determine the position of the second catheter relative to a positional sensor disposed on the first catheter, wherein the positional sensor is disposed a predetermined distance from a distal end portion of the first catheter.

19. The method according to claim 16, wherein analyzing the pattern visible within the captured images includes analyzing a pattern selected from the group consisting of a one-dimensional barcode, DataMatrix, Maxicode, PDF417, a QR Code, a three-dimensional barcode, and a PM code.

20. The method according to claim 16, wherein analyzing the pattern visible within the captured images includes analyzing the pattern visible within the captured images to determine a position of the second catheter relative to a positional sensor disposed at the location on the first catheter.
